(19) United States
(12) Patent Application Publication
(10) Pub. No.: US 2023/0079732 A1
(43) Pub. Date: Mar. 16, 2023

(54) OPAL PEPTIDE ADMINISTRATION

(71) Applicants: THE UNIVERSITY OF SYDNEY, Sydney, NSW (AU); SYDNEY LOCAL HEALTH DISTRICT, Campsie, NSW (AU)

(72) Inventors: Victoria Carroll COGGER, Bexley North, NSW (AU); David George LE COUTEUR, Ryde, NSW (AU); Nicholas HUNT, West Ryde, NSW (AU)

(21) Appl. No.: 17/758,961

(22) PCT Filed: Jan. 18, 2021
(86) PCT No.: PCT/AU2021/050027

§ 371 Date: Jul. 15, 2022

(30) Foreign Application Priority Data
Jan. 17, 2020 (AU) 2020900129

(51) Int. Cl. A61K 47/69; A61K 47/52; A61P 3/10

(52) U.S. Cl. CPC A61K 47/6923; A61K 47/52; A61P 3/10

(57) ABSTRACT

The present invention relates to compositions and methods facilitating the non-invasive administration (such as oral administration) of therapeutic proteins or peptides (such as insulin) which maintain biological activity when absorbed. The composition of the present invention comprises a therapeutic amount of a conjugate, wherein the conjugate comprises a quantum dot and a therapeutically effective peptide or protein.

### RELATED APPLICATION

This application claims priority from Australian Provisional Patent Application No. 2020900129 filed on 17 Jan. 2020, the entire content of which is hereby incorporated by cross-reference.

### FIELD

This invention relates to compositions and methods for administering therapeutic doses of polypeptides and proteins, such as insulin, to a subject via an oral route.

### BACKGROUND

There are a number of diseases or conditions in which a subject is unable to produce sufficient peptides to maintain health. These conditions include, for example, insufficient human growth hormone leading to impaired growth and development, and lack of insulin leading to diabetes. There are numerous examples of other conditions arising from impaired peptide production in mammals.

Type I diabetes mellitus, also referred to as insulin-dependent diabetes or juvenile diabetes, is a chronic condition characterised by the inability of the pancreas to produce insulin. Insulin is a hormone that is produced solely by the islet cells of the pancreas to regulate the amount of glucose in the bloodstream. In a non-diabetic subject, when glucose levels in the bloodstream rise, insulin is secreted by the pancreas into the bloodstream, causing an uptake of glucose by the systemic cells, such as the muscles and liver where it is used as energy or stored as glycogen, and hence out of the bloodstream. As blood glucose levels decrease, so too does the secretion of insulin.

A subject diagnosed with type I diabetes cannot produce insulin and therefore cannot regulate the amount of glucose in their bloodstream. Excessively low and high levels of blood glucose can both have serious health implications. Whilst type I diabetes subjects may be able to limit the variation of glucose in the blood through non-pharmaceutic interventions such as monitoring macronutrient intake and limiting foods high in simple carbohydrates, insulin is still required for the regulation of glucose and the uptake of glucose into cells, especially after meals.

Type II diabetes mellitus generally occurs in adults, particularly those who are overweight. In the early phases of the disease, type II diabetes is associated with insulin resistance and increased circulating levels of insulin. Various oral and injectable medications are used to treat type II diabetes mellitus. Many people with longstanding type II diabetes mellitus eventually require insulin therapy too as insulin production becomes insufficient to regulate systemic metabolism.

Diabetic patients typically need to monitor their blood glucose frequently throughout the day and either ingest glucose if blood levels are too low, or administer insulin parenterally, usually by invasive subcutaneous injection, if blood levels are too high. As insulin is a protein and relies upon its tertiary structure for recognition by insulin receptors, it cannot currently be delivered orally. This is because peptides such as insulin found in the digestive tract are too large to be absorbed and enter systemic cells intact, and the harsh environment due to low pH and proteases in the digestive tract destroy this structure and render the insulin ineffective. Proteins are also not typically absorbed in the intestines with their tertiary structure intact.

One approach has been to provide a formulation of insulin that includes excipients such as chloroquine to delay degradation of insulin in the stomach, as well as absorption enhancers, antioxidants and binders. However, such formulations tend to have slow absorption in the intestine, leading to overly long periods of action, which in some cases can result in insulin activity longer than commercially available “long acting” injections. Such extended activity profiles may make it more difficult for the subject to maintain a constant blood glucose level, and/or may have an extended initial activity period, making such formulations less able to quickly treat a hyperglycaemic episode.

Another approach to delivering insulin to avoid the gastrointestinal tract is to formulate the insulin to be absorbed transdermally across the oral and nasal membranes, in a dosage form such as a spray, chewing gum or lozenge. However, such formulations typically require the insulin to be protected from the environment, such as within a microemulsion formulation, and to include excipients such as buffers, penetration enhancers and stabilizers. Such formulations also typically require refrigerated storage, to ensure the integrity of the insulin. It should also be noted that absorption through the oral and nasal membranes avoids hepatic metabolism.

Another approach has been to tether the insulin to nanoparticles for oral delivery. Such nanoparticles have previously been made out of biopolymers. In one such example, chitosan which had been functionalised to be in the form of a quaternary ammonium salt was used as the core, and then coated with hyaluronic acid to absorb to the mucus layer of the intestine has been described. However, these nanoparticles are too large (approximate average size of about 120 nm) to allow absorption of the nanoparticle per se, instead requiring release of the insulin in the intestine for use. In another such example, gold nanoparticles have been described to attach thiol-containing proteins to the gold surface. However, these nanoparticles are most effective as transdermal agents for absorption across the oral and nasal mucous membranes, with poor intestinal absorption recorded. As noted below, sublingual absorption avoids hepatic first pass metabolism.

While the discussion above relates to insulin as a primary example of a protein- or peptide-based outpatient therapy, the problems existing in the current state of the art relating to insulin delivery also exist for other proteins and peptides that may be therapeutically beneficial to a subject if formulated for oral administration.

Therefore, there is a need for compositions and methods facilitating the non-invasive administration of therapeutic proteins or peptides (e.g. insulin) which maintain biological activity when absorbed. It is desirable for the compositions and methods to be capable of oral administration allowing absorption by the intestine and entry into intestinal blood vessels. This may provide a means to overcome existing problems encountered during oral delivery of proteins and peptides such as insulin. Furthermore, in some specific applications it can be desirable for such compositions and methods to act primarily on the liver, the primary target for insulin action, which may reduce circulating systemic levels of insulin and limit adverse effects such as hypoglycaemia and weight gain.

### SUMMARY OF INVENTION

The present invention aims to alleviate at least one existing deficiency in existing approaches for treating conditions arising from insufficient endogenous peptide or protein production in subjects, and/or orally administering proteins and peptides to treat such conditions.

In a first aspect of the present invention, there is provided a composition comprising a therapeutic amount of a conjugate, wherein the conjugate comprises a quantum dot and a therapeutically effective peptide or protein. The peptide or protein may be less than about 30 kDa in size. It may be between 1 and 25 kDa, or between 3 and 25 kDa, 10 and 20 kDa, 5 and 30 kDa, or 15 and 30 kDa, i.e., it may be about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 kDa. It may have a primary amine group available for attachment to the QD. It may be selected from the group consisting of insulin, growth hormone, fibroblast growth factor 21 (FGF21), a glucagon-like peptide-1 (GLP-1) agonist, a GLP-2 receptor agonist, a platelet derived growth factor (PDGF) beta receptor modulator, an integrin alpha-4/beta-7 antagonist, a PYY(3-36) analogue, vasopressin, interleukins (less than 30 kDa in size), enkephalins, endorphins or any other suitable protein or peptide, and combinations thereof. The insulin may be a native insulin (such as porcine-origin native insulin) or it may be a long-acting analogue (such as glargine insulin or detemir insulin), an intermediate-acting analogue (such as isophane insulin or Neutral Protamine Hagedorn insulin), or a fast-acting analogue (such as insulin aspart, insulin lispro or insulin glulisine). The GLP-1 agonist may be selected from liraglutide or exenatide or it may be any other suitable GLP-1 agonist. The GLP-2 agonist may be apraglutide or it may be any other suitable GLP-2 agonist. The PDGF beta receptor modulator may be BOT191 (also known as Fibroferon), or it may be any other suitable PDGF beta receptor modulator. The integrin alpha-4/beta-7 antagonist may be PN-10943 or it may be any other suitable integrin alpha-4/beta-7 antagonist. Other suitable peptides and proteins that are less than 30 kDa and comprise at least one primary amine group may be known to the skilled person and would be suitable for QD conjugation.

The following options may be used in conjunction with either the first or second aspect, either individually or in any suitable combination.

The composition may be formulated for oral administration.

The quantum dot may be an Ag₂S quantum dot. The average diameter of the quantum dot may be between about 1 nm and about 20 nm. It may be about 1, 2, 3, 4, 5, 6, 7, 8, 9, 10, 11, 12, 13, 14, 15, 16, 17, 18, 19 or 20 nm. The average diameter of the quantum dot may be less than about 10 nm.

The conjugate may further comprise a polymer. The polymer may be a biopolymer. The biopolymer may cover at least part of the conjugate or it may cover substantially all of the conjugate. The biopolymer may be selected from the group consisting of heparin, gelatin, hyaluronic acid, chitosan, galactose, glucose, and any combination thereof. The polymer or the biopolymer may cover at least part of the conjugate. These polymers or biopolymers may protect the protein or peptide of the conjugate during transit through the gastrointestinal tract or specifically target the hepatocytes in the liver. The conjugate may accumulate in or on the surface of a hepatocyte of the subject after oral administration.

The composition may be for administration to a subject that may have been diagnosed with a condition related to insufficient endogenous peptide production. The condition may be type I or type II diabetes or another condition related to insufficient endogenous peptide production. The composition may be for administration to a subject that may have been diagnosed with a condition requiring treatment by an exogenous (i.e., not naturally occurring) protein or peptide. The condition may be diabetic nephrology, liver fibrosis, non-alcoholic steatohepatitis (NASH), renal fibrosis, celiac disease, inflammatory bowel disease (IBD), ulcerative colitis or another gastrointestinal disease. The condition may be treated or treatable by delivery of the protein or peptide to the liver, small bowel, kidneys, pancreas or other gastrointestinal organ or tissue.

The composition may further comprise a pharmaceutically acceptable excipient.

In a second aspect of the present invention, there is provided a method of treating hyperglycemia in a subject in need thereof, the method comprising administering to the subject a therapeutic amount of a conjugate comprising a quantum dot and insulin.

In a third aspect of the present invention, there is provided a method of treating insufficient endogenous peptide production in a subject in need thereof, the method comprising administering to the subject a therapeutic amount of a conjugate comprising a quantum dot and a protein or peptide effective in replacing the insufficient endogenous peptide.

In a fourth aspect of the present invention, there is provided a method of treating a subject suffering from a condition, wherein the condition is treatable with administration of a therapeutic exogenous peptide or protein, the method comprising administering to the subject a therapeutic amount of a conjugate comprising a quantum dot and the therapeutic exogenous peptide or protein.

The following options may be used in conjunction with either the second, third or fourth, either individually or in any suitable combination.

The conjugate may be administered to the subject orally. The subject may have been diagnosed with diabetes or another condition resulting in insufficient amounts of an endogenous peptide or protein in the tissues of the subject. The diabetes being treated in the subject may be type I or type II diabetes.

The quantum dot may be an Ag₂S quantum dot. The average diameter of the quantum dot is between about 5 nm and about 20 nm, or it may be about 1, 2, 3, 4, 5, 6, 7, 8, 9, 10, 11, 12, 13, 14, 15, 16, 17, 18, 19 or 20 nm.

The conjugate may further comprise a polymer. The polymer may be a biopolymer. The biopolymer may at least partially coat the conjugate, or it may substantially coat the conjugate. The biopolymer may be selected from the group consisting of heparin, gelatin, hyaluronic acid, chitosan, galactose, glucose, and any combination thereof.

In a fifth aspect of the present invention, there is provided a method of delivering a peptide or protein to an organ of a subject, the method comprising orally administering a conjugate comprising a quantum dot and the peptide or protein to the subject, wherein the organ is selected from the liver, pancreas, small bowel or kidneys.

The following options may be used in conjunction with the fifth aspect, either individually or in any suitable combination.

The quantum dot may be a Ag₂S quantum dot. The Ag₂S quantum dot may be between about 5 nm and about 20 nm in diameter.

The conjugate may optionally comprise a polymer. The polymer may be a biopolymer. The biopolymer may be selected from the group consisting of gelatin, chitosan, galactose, glucose, and any combination thereof.

In a sixth aspect of the present invention, there is provided a method of lowering blood glucose in a subject, the method comprising orally administering a conjugate comprising a quantum dot and insulin as described herein to the subject, wherein the quantum dot is an Ag₂S quantum dot between about 5 nm and about 20 nm, and wherein the conjugate is metabolized in or bound to the surface of a hepatocyte, thereby releasing the insulin into the blood stream of the subject

In a seventh aspect of the present invention, there is provided use of a conjugate comprising a quantum dot and insulin as described herein for the manufacture of a medicament for the treatment of type I and type II diabetes. The medicament may be formulated for oral administration. The medicament may further comprise a polymer or a biopolymer.

In an eighth aspect of the present invention, there is provided use of a conjugate comprising a quantum dot and a protein or peptide in the preparation of a medicament. The medicament may be for treating insufficient endogenous production of the peptide or protein in a subject or it may be for providing a therapeutically active exogenous peptide or protein. The medicament may be formulated for oral administration. The medicament may further comprise a polymer or a biopolymer.

### BRIEF DESCRIPTION OF DRAWINGS

Preferred embodiments of the present invention will now be described, by way of example only, with reference to the accompanying figures, wherein:

FIG. 1 shows a schematic representation of a method for synthesizing and characterising water soluble Ag₂S quantum dots of the present invention. Specifically: (a) Schematic illustrating production of cyclohexane soluble QDs. (b) NIR II emission at 1175 nm from 658 nm excitation. (c) FTIR spectra demonstrating dodecane C—H chains on the surface of QDs. (d) TEM images of QDs showing arrangement, size and lattice structure. (e) schematic of phase transfer of COOH capped QDs with representative samples of 1 mM QDs in organic solvent and water. (f) FTIR spectra showing OH functional group with C═O, C—O and OH fingerprints. (g) TEM images of monodispersed QDs in water.

FIG. 2 shows protein corona and synthetic surface topology alterations to quantum dots. Specifically: (a) FTIR of water soluble QD, (b-d) formation of the protein corona following 24 hr incubation with (b) RPMI media, (c) mouse serum and (d) BSA. Protein corona demonstrates formation of amine functional groups (amine I and II). (f-g) synthetic polymer deposition on QD surface topology. Grey insert boxes show raw material FTIR spectra with grey arrows showing OPUS wizard identified determinant peaks. Binding of biopolymer to QDs was performed using click chemistry (EDC/NHS coupling). QD-heparin (e) and QD-gelatin (f) all demonstrated similar FTIR spectra to the raw biopolymer. Black arrows show OPUS wizard identified determinant peaks, which corresponded to peaks shown in the inserts. Peaks identified were amine I, II and III.

FIG. 3 shows the lifecycle of Ag₂S quantum dots following oral gavage administrations. Specifically, (a) Time lapse images of QD-488 following injection into the lumen of the small intestine in an anesthetized mouse. Following injection, the small intestine demonstrates strong 488 florescence compared to blood vessels (t=0-3 mins). At 5-6 mins the blood vessels demonstrate 488 florescence compared to the surrounding tissue; progressive reduction in 488 fluorescence is observed in the small intestine up to 10 mins post injection. (b) Biodistribution of ³H-QDs was measured at 30 mins, 2 hrs and 24 hrs post gavage of 100 μl of 1 mM QDs. 30 mins post gavage the liver demonstrated 60% accumulation of the ingested radioactive dosage with minimal expression in other organs. 2 hrs post gavage the liver demonstrated 40% expression of the ingested radioactive dosage. Over the 24 hr period post gavage fecal matter demonstrated 80% of the ingested radioactive dosage with minimal expression observed remaining in the organs. (c) Liver function tests were performed using AST and ALT assays using blood samples collected 24 hrs post gavage. (d) H&E stained tissue samples from the liver kidney, small intestine and spleen demonstrated no immune cell infiltration or cellular necrosis.

FIG. 4 shows blood glucose levels in healthy rodents that were administered a glucose gavage 30 minutes after a treatment with either an oral gavage of quantum dot-insulin conjugates equivalent to a 20 IU/kg dose of insulin, or intraperitoneal injection of active insulin equivalent to a 0.1 IU/kg dose of insulin and were compared to a control group that was not given any insulin therapy. Each subject was then monitored for blood glucose over the 90 minutes after receiving the glucose gavage. The graphs show that both the oral conjugate dose and intraperitoneal injection reduced the blood glucose of the rodent subjects by an approximately equivalent amount and reduced the spike in blood glucose seen in the control group.

FIG. 5 shows blood glucose levels in 3 month old healthy C57BL/6 mice (n=5) were given either: a sham subcutaneous injection of saline (Control), a subcutaneous injection of insulin (SC-INS, 2 IU/kg), or an oral gavage of oral-insulin (QD-INS, 20 IU/kg), 30 mins prior to an oral glucose tolerance test (oGTT). oGTTs were performed using a glucose bolus oral gavage (2 g/kg) and blood sampling at −15, 0, 15, 30, 45, 60 and 90 mins from a tail snip. Data in this figure shows the mean±SD and the area under the curve (AUC).

FIG. 6 shows the pharmacological biodistribution of ¹⁴C radiolabelled insulin when given by subcutaneous injection or oral gavage in 3 month old healthy C57BL/6 mice (n=3) which were given either: a subcutaneous injection of insulin (SC-INS, 2 IU/kg), an oral gavage of oral-insulin (QD-INS, 2 IU/kg) or an oral gavage of insulin (oral-INS, 2 IU/kg), 0.5 or 2.0 hrs prior to euthanasia and isolation of whole blood and whole organs (liver, kidney, spleen and small bowel). Tissue samples and blood plasma were prepared for radiolabel component analysis according to standard methods. Data is presented in this figure is as a percentage of radiolabel insulin relative to the administrated dosage, data is shown as mean±SD.

FIG. 7 shows pharmacodynamic (PD) and pharmacokinetic (PK) effects of subcutaneous and oral insulin in 3 month old healthy C57BL/6 mice (n=3) which were given either: a sham subcutaneous injection of saline (Control), a subcutaneous injection of insulin (SC-INS), or an oral gavage of oral-insulin (QD-INS) 0.5 hrs prior to an oral glucose tolerance test (oGTT). As shown in FIG. 5, insulin treatment promotes a reduction in the AUC of the GTT. PD data was collected using 0.5, 1 and 2 IU/kg SC-INS or 10, 20, 30, 40, 50 and 100 IU/kg QD-INS. Data shows the % effect size on the oGTT AUC relative to 2 IU/kg SC-INS (i.e. relative to the effect size shown by SC-INS in FIG. 4). PK/PD data was collected using data generated from oGTTs performed at 0.5, 1.0 and 2.0 hrs post administration of sham, 2 IU/kg SC-INS or 20 IU/kg QD-INS. Data shows the percentage reduction in the oGTT AUC with data points showing the mean±SD.

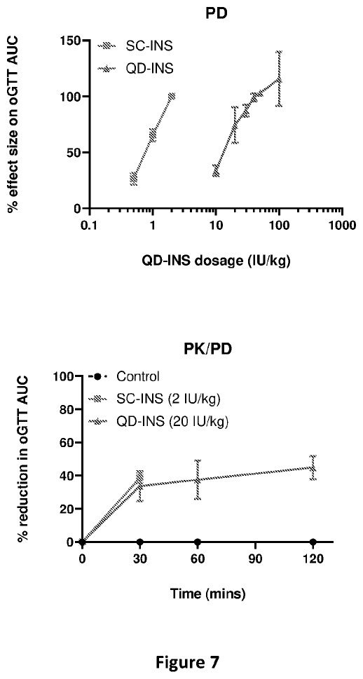

FIG. 8 shows insulin tolerance testing in 4 month old NOD/Scid mice (n=3) using subcutaneous (SC-INS) and oral (QD-INS) insulin. The mice demonstrated development of diabetes with increasing blood glucose concentrations. NOD mice with a blood glucose below 30 mg/dl were treated with SC-INS (1 IU/kg) or QD-INS (25 IU/kg) at time 0, mice with a blood glucose above 31 mg/dl were treated with 4 IU/kg SC-INS or 100 IU/kg QD-INS. Blood glucose samples were collected at −15, 0, 15, 30, 45 and 60 mins. Data in this figure shows the change in blood glucose relative to the initial blood glucose concentration. Data points showing the mean±SD.

FIG. 9 shows in vivo toxicity of Ag₂S QDs in WT C57BL/6J mice of 3-4 months of age occurs at 256 μg/ml, compared to the therapeutic dose of QG-insulin (25.6 ng/ml).

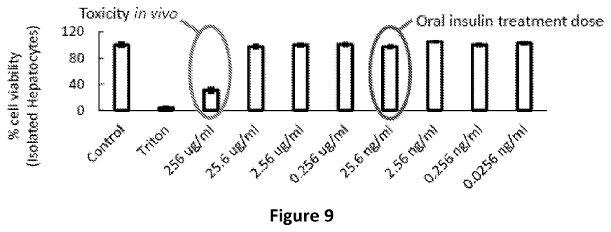

FIG. 10 shows the effect of QD-liraglutide conjugates on (a) blood glucose levels and (b) body weight in WT C57BL/6J mice at 15 months of age. Blood glucose levels (FIG. 10a)) were taken 30 minutes after an intraperitoneal injection 250 μg/kg of liraglutide, or two hours following oral gavage of QD-liraglutide at either 1250 μg/kg or 2500 μg/kg. Body weight (FIG. 10b)) was measured following 3 treatments per week of 2500 μg/kg oral QD-liraglutide.

FIG. 11 shows the distribution of a 100,000 DPM dose of oral ¹⁴C-labelled metformin, ¹⁴C-labelled QD-metformin conjugates (NP-metformin), and ¹⁴C-labelled QDs (NP) in WT C57BL/6J mice at 3-4 months of age (n=3 per treatment group) over 24 hours post-administration.

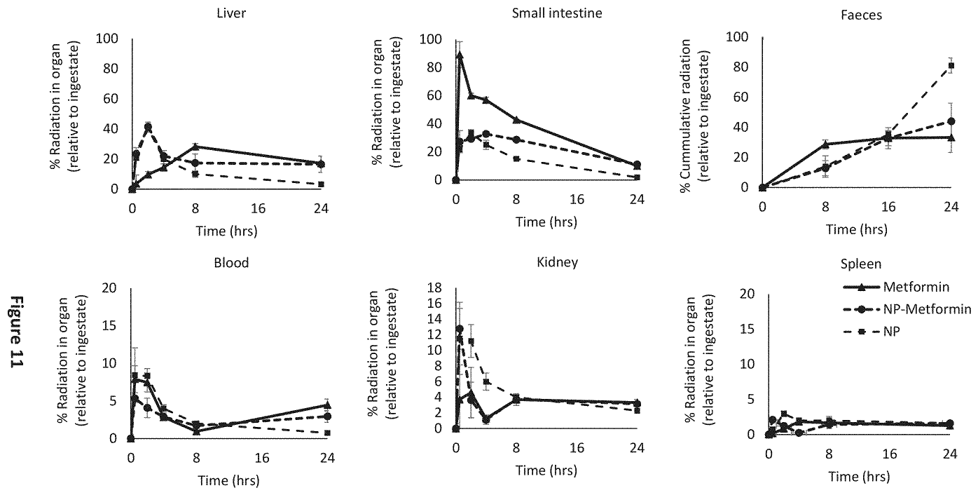

### DEFINITIONS

The following definitions are provided to enable the skilled person to better understand the invention disclosed herein. These are intended to be general and are not intended to limit the scope of the invention to these terms or definitions alone.

The term “blood glucose” as used herein refers to the amount of glucose circulating in the blood of the circulatory system of a subject. Accordingly, the related terms “lowering blood glucose” or “decreasing blood glucose” and the like should be understood to refer to less glucose circulating in the blood of the subject compared to an earlier time, which may be before administration of a treatment. Likewise, terms such as “raising blood glucose” or “increasing blood glucose” and the like should be understood to refer to more glucose circulating in the blood of a subject compared to an earlier time.

The terms “hyperglycaemia” and “hyperglycaemic” and the like refers to when the blood glucose level of a subject is higher than the normal range for blood glucose in a healthy subject. Similarly, the terms “hypoglycaemia” or “hypoglycaemic” and the like refers to when the blood glucose levels of a subject are lower than the normal range for blood glucose in a healthy subject.

The term “quantum dot” as used herein refers to a nanoparticulate material with an average size distribution of approximately less than 20 nm. Such quantum dots also generally exhibit optical and/or electronic properties that are distinct from particles formed of the same material but of a larger size.

The term “conjugate” as used herein refers to an arrangement whereby two or more species and/or structures are in association. For example, a quantum dot of the present invention may be associated with an insulin protein chain to form a quantum dot-insulin conjugate. Such association may take any suitable chemical form, so long as the two or more conjugated elements are found in close proximity.

The term “polymer” as used herein refers to a molecule or macromolecule formed by linking monomers with covalent bonds. The monomers may be the same or they may be different. The monomers may be formed into repeating subunits of monomers (e.g., polyethylene) or they may have a non-repeating sequence (e.g. a protein). The term “polymer” is understood to encompass polymers of both a synthetic origin and a biological origin.

The term “biopolymer” as used herein refers a polymer chain of biological origin, whereby the polymer is produced by a biological system, and/or in which a polymer chain is formed from monomer moieties which have a biological origin.

The term “peptide” or “polypeptide” as used herein refers to a short chain of amino acids, connected in sequence by peptide bonds, usually between about 2 and about 50 amino acids in length. The term “protein” as used herein refers to a chain of amino acids which are longer in length than peptides, i.e., about 51 amino acids or more in sequence.

The term “oral”, with reference to administration of compositions of the present invention, refers to delivery to the mouth of the subject to whom the composition is being administered, with the expectation that most, if not all, of the composition is absorbed in the gastrointestinal tract. The term “oral” in the context of the present invention is not intended to refer to transdermal absorption across the mucosal membranes.

The term “administration”, or variations including but not limited to “administer” or administering”, as used herein refers to providing a subject with a therapeutic composition.

The terms “treating”, “treatment”, “therapy”, “therapeutic” and the like as used herein refer to the administration of a composition to ameliorate an adverse medical condition or to reduce the symptoms thereof in the subject to whom the composition is administered. Accordingly, in the context of the present invention, the effect of administration of a “therapeutic conjugate” encompasses reducing at least one of the symptoms of type I diabetes, such as reducing blood glucose in a hyperglycaemic subject, which may be administered as a curative therapy or as a prophylactic therapy.

The term “subject” as used herein refers to any human or non-human animal to be treated. Accordingly, compositions of the present invention may be suitable for human treatment, and they may also be suitable for veterinary treatment of non-human animals, including companion animals such as cats and dogs, or farm animals, such as pigs, horses, sheep and cattle.

The term “hydrodynamic diameter” as used herein refers to the degree of arrangement of the water molecules close to the surface when the conjugate is dispersed in water. It is defined as the diameter of a perfect solid sphere that would exhibit the same hydrodynamic friction as the conjugate when dispersed in water. In other words, as the surface of the conjugates described herein have an electrostatic charge, the water molecules adjacent to the surface would be arranged and associated with the surface by electrostatic forces to provide a particle essentially a defined water layer shell, and hence with a hydrodynamic diameter equivalent to a solid particle without such water association.

As used herein, the term “comprising” means “including”. Variations of the word “comprising”, such as “comprise” and “comprises”, have correspondingly varied meanings. As used herein, the terms “including” and “comprising” are non-exclusive. As used herein, the terms “including” and “comprising” do not imply that the specified integer(s) represent a major part of the whole.

As used herein, the term “consisting essentially of” means “to the exclusion of other additional components purposefully added”, or “only the following recited elements are intended to be present”. Additional components that are in the defined composition or device that are not intentionally present are acceptable.

### DESCRIPTION OF EMBODIMENTS

The present invention relates to a composition for providing therapeutic peptides and proteins to a subject in need thereof. The composition may be used in a method for treating insufficient endogenous peptide production in a subject, or it may be used in a method for delivering a therapeutic exogenous peptide or protein to the subject. The subject may be suffering from a condition whereby levels of at least one endogenous peptide is insufficient and requires replacement therapy. One well-known, non-limiting example of a condition where a subject has insufficient endogenous protein production is type I diabetes. Accordingly, in one embodiment, the present invention is directed to lowering blood glucose in a subject, and also to a method for lowering blood glucose in a subject. The subject may be suffering from type I or type II diabetes, in which the subject is unable to produce insulin and so requires therapy to maintain a normal blood glucose level. The subject may be hyperglycaemic and require a therapeutic dose of insulin to lower their blood glucose level. The subject may be suffering from another condition whereby levels of at least one endogenous peptide is insufficient and requires replacement therapy, an example of which is type I diabetes. The subject may be suffering from a condition that requires delivery of a therapeutic exogenous peptide or protein, such as for example: a PDGF receptor beta modulator in the treatment of diabetic nephrology, liver fibrosis, non-alcoholic steatohepatitis (NASH) and renal fibrosis; a GLP-2 receptor agonist in the treatment of celiac disease and other gastrointestinal diseases; or integrin alpha-4/beta-7 antagonists in the treatment of inflammatory bowel disease (IBD) and ulcerative colitis. The composition of the present invention can advantageously deliver the therapeutic endogenous or exogenous peptides and proteins directly to the organ or cells affected by the condition, and/or to an organ that is capable of releasing the peptide or protein, in a process that is analogous to natural processes for distributing endogenous peptides and proteins.

In one embodiment, a composition of the present invention comprises a conjugate of insulin with a quantum dot. The inventors have surprisingly found that such conjugates can enter the circulatory system of the subject after oral administration and absorption through the intestinal wall. The quantum dots then deliver the insulin to the hepatocytes via the portal circulation where the insulin activates the insulin receptor then the conjugate is taken up by the hepatocytes by endocytosis for subsequent metabolism and excretion via the bile. As will be described in greater detail below and with reference to the examples, insulin (as an example of an endogenous protein suitable for replacement therapy) can be effectively administered orally to a subject in order to lower blood glucose levels, however the skilled person would be aware that such compositions and methods as exemplified herein may be expanded to other suitable therapeutic peptides, particularly those that benefit from administration to the liver.

### Insulin

Insulin is a peptide hormone produced by and secreted from the beta, or islet of Langerhans, cells found in the pancreas of a healthy subject. In a healthy subject, insulin secretion usually occurs in response to a high blood glucose level in a subject, acting to cause the cells of the subject to take up glucose out of the blood system, with excess glucose being either polymerised and stored as glycogen in the liver and muscles, or converted into fatty acids for storage in the adipose tissues as fat.

Structurally, insulin is as a dimer of two chains linked via disulfide bonds, the dimer referred to herein as an insulin molecule. As would be understood by the skilled person, the resulting quaternary structure is important for the activity of insulin to bind to the insulin receptors embedded in the membranes of cells, with any disruption of the disulfide bonds or cleavage of the peptide chains likely to result in little to no activity. As insulin administration is currently the only therapy available to treat type I diabetes and is also a major treatment option for people with type II diabetes mellitus, the administration of insulin in a manner that maintains the integrity of the quaternary structure of the insulin molecule is important.

Commonly, the insulin that is currently used in the treatment of humans is human insulin that is produced by use of recombinant DNA technologies. Insulin from other species, such as from pigs, has also been used previously to treat humans. However, the amino acid sequence and resulting structure of insulin differs slightly between species. For example, porcine-origin insulin differs by one amino acid residue compared to the human-origin sequence, and bovine-origin insulin differs by three amino acid residues compared to the human-origin sequence. These slight differences mean that the use of insulin harvested from another species is unlikely to be as effective as insulin produced by cells of the same species. However, cross-species administration can still be effective in therapy. For instance, before recombinant DNA techniques were available, insulin was regularly harvested from pigs for use in human therapy, due to the closeness of the two amino acid sequences. Insulin derivatives are also available with varying lengths of activity. For instance, insulin is available as a long-acting analogue (such as glargine insulin or detemir insulin), an intermediate-acting analogue (such as isophane insulin or Neutral Protamine Hagedorn insulin), or a fast-acting analogue (such as insulin aspart, insulin lispro or insulin glulisine).

Accordingly, the present invention is not limited to the use of an insulin molecule of any particular amino acid sequence or species of origin. By “species of origin”, it is meant that the insulin used herein has the same sequence as that produced naturally by healthy members of that origin species. The “species of origin” does not need to be the same as the subject species. The insulin may be of a sequence that is the same or similar to the native sequence of the subject species, or it may be a different species with a quaternary protein structure that is effective in treatment of the subject. The amino acid sequence is preferably native (i.e., as produced by the cells of the subject species), but it may also be chemically modified, so long as any such modification does not significantly affect the efficacy of the insulin in the subject after administration.

The insulin used in the present invention may be produced by any suitable method. For example, it may be made, collected and purified from use of recombinant DNA technology or it may be harvested from an animal or a cultured collection of mammal cells that express insulin or it may be made by chemical synthesis. It may be produced as a salt. It may be used herein as a pharmaceutically acceptable salt of insulin. It may be a long-acting, intermediate-acting, or short-acting insulin derivative.

### Quantum Dot

The conjugates used in the present invention comprise a nanoparticle referred to herein as a quantum dot. By definition, a quantum dot is a nanoparticle of relatively small size, up to about 50-100 nm in diameter, but which displays electronic and/or optical properties (or “optoelectronic” properties as used interchangeably herein) that are different compared to particles formed from the same matter but of larger size or to the bulk material. An example of the optical properties of quantum dots that may be observed is photoluminescence, whereby a wavelength in the ultraviolet portion of the electromagnetic spectrum is absorbed by the quantum dot, exciting an electron into a higher energy level which then degrades to a lower energy electron shell, releasing a quantum of energy which is observed as a wavelength in the visible portion of the electromagnetic spectrum. An example of the electronic properties of quantum dots that may be observed as superconductivity. These optoelectronic properties of quantum dots may be dependent on particle size and the materials used in their construction. In the context of the present invention, the optoelectronic properties of quantum dots may be used diagnostically to determine the location of the conjugate in different tissues of the subject following administration of a composition comprising these conjugates with suitable imaging techniques.

The quantum dots may be formed from any suitable material. By “suitable”, it is meant that the quantum dot must comprise a material, or materials, which are capable of forming a particle small enough to be characterised as a quantum dot, which display the optoelectronic properties characteristic of a quantum dot, and which are capable of associating with a protein, either on the native material surface or after functionalisation. By “functionalisation”, it is meant that the surface is changed following a chemical reaction. As they are for therapeutic administration to a subject, the quantum dots of the present invention must also be non-toxic to that subject and well-tolerated. As they are for treatment of a chronic condition, they must also be substantially eliminated soon after therapy (i.e., do not accumulate in the body, tissues or cells of the subject). In this regard, “substantial elimination” may mean that greater than about 75% of the quantum dots that are administered are eliminated from the subject in a given time. Preferably, about 75% of the quantum dots would be eliminated from the subject within 48 hours, within 24 hours or within 12 hours after administration.

They may be core-type quantum dots that are formed from a single material, such as chalcogenides (e.g., selenides, sulfides or tellurides) of non-heavy metals, such as zinc or silver. They may be formed from a material which comprises or consists of Ag₂S, ZnS or any other suitable material. They may be formed from Ag₂S, which is understood to be non-toxic to mammals. They may be core-shell quantum dots that are formed from two different materials whereby a layer of higher band gap superconducting material is coated onto a core. The shell layer must consist of a non-toxic material. A common example of a core-shell quantum dot is a shell layer that consists of CdSe applied onto a core comprising ZnS, although this layer material would not be suitable for use in the present invention due to the presence of cadmium, a known toxic heavy metal. They may be alloyed quantum dots, whereby two or more semiconducting materials are combined by alloying. Alloyed quantum dots may result in properties distinct from the bulk properties of either of the constituent materials. The quantum dots may be crystalline. Any of these types of quantum dots may be suitable for use in the present invention.

It is known that quantum dots may be produced, fabricated or prepared using several different methods. The quantum dots of the present invention may be produced by any suitable method currently known or may be developed. For example, they may be produced by colloidal synthesis, plasma synthesis, self-assembly or electrochemical assembly.

There appears to be some disagreement in the field of nanomaterials as to the accepted upper size limit of a quantum dot, as distinct from a nanoparticle, in terms of average particle diameter. In some published literature in this field, a quantum dot is defined as less than 20 nm; in other literature a quantum dot is defined as less than 50 nm; and in yet other literature a quantum dot is defined as between 1 nm and 100 nm. The quantum dots of the present invention are generally defined herein as particles with a diameter of less than 20 nm. Accordingly, each quantum dot disclosed herein may have a diameter of between about 1 nm and about 20 nm, or they may be between about 1 nm and 5 nm, 5 nm and 10 nm, 10 nm and 20 nm, 5 nm and 15 nm, 1 nm and 15 nm or 5 nm and 20 nm, e.g., they may have a diameter of about 1, 2, 3, 4, 5, 6, 7, 8, 9, 10, 11, 12, 13, 14, 15, 16, 17, 18, 19 or 20 nm. As will be discussed below in further detail, this upper size limit is due to the restrictions inherent in the processes of absorption through the intestinal wall and endocytosis in the hepatocytes of the liver, which are believed to be steps involved in the therapeutic effects provided by the present invention, rather than any inherent limit placed on the material itself. Accordingly, quantum dots larger than 20 nm, up to 50 nm or up to 100 nm, e.g., average diameters of up to 20, 25, 30, 35, 40, 45, 50, 55, 60, 65, 70, 75, 80, 85, 90, 50 or 100 nm may be suitable for use in the present invention.

The quantum dots may be any suitable shape. They may be spherical or mostly spherical. They may be irregularly shaped. A population of quantum dots may all have a uniform shape, or there may be more than one shape in any population.

### Silver Sulfide Quantum Dots

In one embodiment, the quantum dots of the present invention may consist of or comprise Ag₂S. Quantum dots that consist of Ag₂S are well known in the field of nanoparticles. They have been shown to have low or no toxicity to mammals and may also have near-infrared fluorescence. Commonly, they are prepared by using a self-assembly method which results in Ag₂S quantum dots that have a hydrophobic coating, however the Ag₂S quantum dots of the present invention may be made by any suitable method. Such hydrophobic Ag₂S quantum dots are commonly functionalised to have a hydrophilic coating. This functionalisation may be carried out with use of any suitable method to obtain a hydrophilic coating. In one example, the hydrophilic reagent used may be a mercapto- or thiol-containing reagent which, when incubated with the hydrophobic quantum dots in suitable conditions, results in a hydrophilic surface chemistry on the Ag₂S quantum dots. The surface of the Ag₂S quantum dot may be completely or substantially covered in polar functional groups, such as for example carboxyl, hydroxyl, thiol or amino functional groups. Functionalisation to form a hydrophilic surface on the Ag₂S quantum dot usually occurs in a polar solvent. The resulting hydrophilic Ag₂S quantum dots are then usually stable, do not aggregate together and can be associated with insulin to form a conjugate suitable for therapy.

### Quantum Dot-Insulin Conjugates

The present invention includes the use of therapeutic conjugates. All conjugates referred to herein are stable associations formed between a quantum dot and at least one insulin molecule. Standard conjugation chemistry may be used to conjugate the insulin molecule(s) to the surface of the quantum dot. In one such example, a therapeutic conjugate may be formed via the steps of contacting a suitable quantum dot, which may be hydrophilic or hydrophobic, with a coupling agent and insulin in a solvent and then incubating the mixture to form a crude therapeutic quantum dot conjugate. The contacting of these three reagents may occur simultaneously, whereby all reagents are added together and incubated, or they may occur sequentially, whereby two of the three reagents are added together, such as for example the quantum dots and the coupling agent, and the reaction completed, before the third reagent is then added. The crude therapeutic quantum dot conjugate may then optionally be purified by any suitable method to remove the unreacted reagents. One such method may be a solid state purification method whereby several cycles of washing with a suitable solvent and then filtering are performed. Another method may be that the conjugates are separated from the solvent by centrifugation and then used a part of in a composition.

Coupling agents may be used to form an amide linker group or an ester linker group between the polar functional groups bound to the surface of the quantum dot and the conjugated insulin molecule, although the skilled person would understand that more than one bond may be formed between the quantum dot surface and the insulin. In one example, the quantum dot may be functionalised so as to exhibit carboxyl groups on its surface, which may be linked to either a carboxyl or amino group of the insulin via an amide or ester linker group. Coupling agents that are known and may be used include benzotriazolyloxytris(dimethylamino)phosphonium hexafluorophosphate (BOP), carbodiimides such as dicylcohexylcarbodiimide (DCC), diisopropylcarbodiimide (DIC) and 1-(3-dimethyl-aminopropyl)-3-ethylcarbodiimide hydrochloride (EDC), N-hydrosuccinamide and sulfo-N-hydroxysuccinamide (NHS), although any suitable coupling agent may be used.

Proteins such as insulin can be sensitive to environmental conditions, such as temperature, shear, pH, salt concentration and solvent. Depending on the protein, some conditions may cause irreparable damage to the structure of the protein, in some causes causing denaturation, rendering it ineffective. Accordingly, when forming quantum dot conjugates, the processing conditions should be carefully considered to ensure that the insulin remains effective after conjugation with the quantum dot.

The solvent that the coupling reaction takes place in may be polar or it may be non-polar, depending on the chemistry of the quantum dot surface and reagents involved. The solvent may be water. The solvent may be a protic polar organic solvent, such as methanol, ethanol, butanol, or propanol. The solvent may be an aprotic polar organic solvent, such as acetone, acetonitrile or N,N-dimethylformamide (DMF).

When the solvent is water, salts may be added or be present so as to approximate biological osmolarity and maintain the integrity of the insulin molecules. The conjugation reaction may be carried out in a saline environment, whereby the saline is water with dissolved salts such as NaCl and KCl present. The saline environment may estimate plasma osmolarity of between about 300 and 312 mOsm/L. A buffer may also be added to the water or saline to ensure the pH of the reaction mixture does not cause the denaturation of the insulin during the conjugation reaction. The buffer may maintain a biological pH of between about pH 6 and about pH 8. The buffer may be, for example, a phosphate buffer, a Tris buffer, a citrate buffer or a glycine buffer, or any other suitable buffer. A buffer is not generally required in an organic solvent phase, as hydronium ions are not usually present, therefore pH is not a factor.

The temperature that the reaction occurs at may be mild in any step that includes insulin as a reagent. The temperature may be between about 1° C. or about 40° C., or it may be between 1° C. and 20° C., 10° C. and 30° C., 5° C. and 35° C., 20° C. and 40° C., 15° C. and 25° C. or 25° C. and 35° C., e.g., it may be at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40° C. It may be at a room temperature. The temperature may be consistent throughout the reaction, with variation of between 1 and 5° C. between maximum and minimum temperatures, or it may vary throughout, such as on a heating gradient of a cooling gradient. It would be expected that the minimum temperature was greater than about 1° C., and the maximum temperature is less than about 40° C., to avoid damage to the insulin that may be caused by freezing (in a low-osmotic water solvent) or heat-induced denaturation respectively. If the reaction is carried out sequentially, the reaction step that includes only the quantum dots and the coupling agent may occur at a higher temperature, such as between 1° C. and 100° C., or between 10° C. and 50° C., 25° C. and 75° C., 15° C. and 85° C., 35° C. and 65° C., 70° C. and 90° C., or 50° C. and 100° C., e.g., it may be at 1, 2, 3, 4, 5, 10, 15, 20, 25, 30, 35, 40, 45, 50, 55, 60, 65, 70, 75, 80, 85, 90, 95 or 100° C.

Each reaction step in the reaction may occur over a time of between about 5 minutes and about 10 hours, or between about 5 minutes and 5 hours, 1 hour and 6 hours, 2 hours and 8 hours, 5 hours and 10 hours, or 30 minutes and 3 hours, e.g., 5 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours or 10 hours. It may be left for a long enough time to ensure that the reagents are completely or substantially completely reacted.

### Biopolymer

As described below in more detail, the quantum dot-insulin conjugates may also comprise a polymer. The polymer may be a synthetic polymer or it may be a biopolymer. This biopolymer may be applied to the quantum dot-insulin conjugates so that the conjugates are at least partially coated in a biopolymer, or the biopolymer may be dispersed with the insulin in an amalgam layer. The conjugates may be completely coated by a biopolymer or they may be substantially coated by a biopolymer. This outer layer comprising a biopolymer may act to protect the insulin, whether by extending shelf life or by forming a physical barrier and reducing degradation after administration. It may allow for, or promote, absorption across a membrane, such as an intestinal membrane if administered orally by swallowing, or across a nasal membrane if administered as a spray. It may allow for targeted absorption by a desired cell type after absorption by the subject after administration. The role of the biopolymer may be suited for any one or more of these roles.

The biopolymer layer may comprise any suitable biopolymer, depending on the desired effect. For instance, if the desired effect is as a physical barrier, the biopolymer must be capable of forming a water-impervious, and possibly an air-impervious, layer around the conjugate.

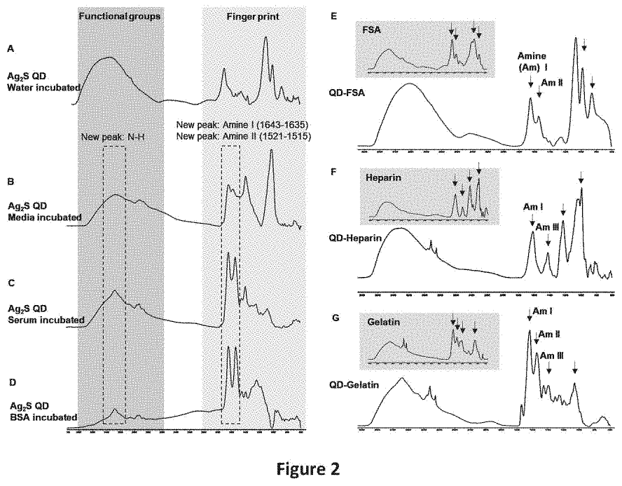

For instance, biopolymers such as gelatin, heparin, or even the absence of a biopolymer coating, may result in conjugates with a relatively small hydrodynamic diameter and hence show a preference for endocytosis of the conjugate into hepatocytes by clathrin-mediated endocytosis. Further, certain biopolymers may protect the insulin-QD conjugates from the acid environment and digestive enzymes of the gastrointestinal tract. For example, biopolymers such as chitosan or galactose or glucose, or a combination of these, may result in the protection of the conjugates from gastrointestinal pH and digestive enzymes. These biopolymers, whilst selected for their barrier properties, may also affect the hydrodynamic radius of the conjugate and hence potentially affect the uptake of these coated conjugates by the cells of the liver. The surface chemistry of the QDs, either with or without a biopolymer coating, can be analysed via FTIR to determine which groups are present (see FIG. 2).

The biopolymer layer may be applied to the conjugates by any suitable process. By way of example, in one such method, biopolymer attachment may be performed by the formation of an amide bridge between carboxylic acid groups on the QDs and a primary amine group on the biopolymer. For example, this reaction may be performed in the presence of N-hydroxysuccinimide (NHS) or its water-soluble analog (Sulfo-NHS) and 1-Ethyl-3-(3-dimethylaminopropyl)carbodiimide (EDC). This reaction requires changes in pH that can be promoted by the addition of HCl and NaOH solutions. Optionally, this method may also use additional linker(s), such as adipic acid dihydrazide.

The thickness of the biopolymer layer, not including interactions at its surface with water molecules, may be between about 5 nm and about 25 nm, such as between about 5 nm and 10 nm, 10 nm and 20 nm, 15 nm and about 25 nm, or 12 nm and 17 nm, e.g., about 5, 6, 7, 8, 9, 10, 11, 12, 13, 14, 15, 16, 17, 18, 19, 20, 21, 22, 23, 24 or 25 nm. The resulting hydrodynamic diameter of the conjugates, with or without biopolymer coating, may be between about 10 nm and about 100 nm, such as between about 10 nm and 50 nm, or between 20 nm and 40 nm, 25 nm and 75 nm, 50 nm and 100 nm, 30 nm and 80 nm, 40 nm and 60 nm, or 60 nm and 100 nm, e.g., about 10, 15, 20, 25, 30, 35, 40, 45, 50, 55, 60, 65, 70, 75, 80, 85, 90, 95 or 100 nm.

### Therapy

The conjugates described herein, comprising a quantum dot and insulin, optionally further comprising a biopolymer, may be used in the treatment of type I or type II diabetes in a subject in need thereof. As described above, insulin is the most effective therapy for insulin-dependent diabetes. These conjugates have been developed by the inventors specifically as a therapy for providing a diabetic subject with insulin when in need, such as in a curative manner during a hyperglycaemic event, or prophylactically before or during a meal or when waking.

Without being bound to theory, it is believed that these quantum dot-insulin conjugates are bound to the cell surface and engulfed by cells of the body of the subject, preferably the liver and more preferably the hepatocytes, where the insulin can act to lower the blood glucose of the subject. Whilst there is a preference for the quantum dot-insulin conjugates to target the liver, they may also be found in other organs, such as the small bowel shortly after oral administration, or in the kidney, spleen and pancreas. The quantum dot-insulin conjugates specifically target the hepatocytes, thereby reducing systemic hyperinsulinemia and associated adverse effects. It is envisioned that these conjugates can be administered to the subject by any suitable means that results in the conjugate entering the bloodstream of the subject and contact with cells that metabolise the conjugates and release the insulin. For instance, the conjugates may be administered by subcutaneous injection, similarly to the common route of administration of insulin known in the art, although oral administration is preferred.

### Oral Administration

One particularly advantageous and surprising route of administration for the delivery of these conjugates is via oral administration. An oral dosage form of insulin would avoid the problems known with subcutaneous injection of insulin (and other proteins and peptides), such as poor compliance by subjects and a build-up of scar tissue at common injection sites making further injections difficult. A composition in an oral dosage form would be expected to increase compliance and reduce unwanted side effects related to subcutaneous administration. By “oral administration”, it is meant that the subject takes the composition orally by swallowing a composition comprising the conjugates for absorption in the gastrointestinal tract, rather than absorption across oral membranes such as the sublingual mucosa.

A composition for oral dosing of insulin comprises a quantum dot-insulin conjugate, optionally with a biopolymer coating. The composition may also include pharmaceutically acceptable excipients, such as diluents (e.g., lactose, dextrin, silicates, magnesium salts, or calcium salts), binders (e.g., starches, cellulose, cellulose derivatives, or sugar alcohols), disintegrants (e.g., starch, cellulose derivatives or alginates), glidants (e.g., colloidal anhydrous silica and other silica compounds), preservatives (e.g., sodium benzoate, EDTA, sorbic acid or parabens), antioxidants (e.g., BHA, BHT, tocopherol acetate) and lubricants (e.g., steric acid and salts thereof). An oral dosage form may be in any suitable physical form, such as for example a tablet, a capsule, a powder, a suspension or a solution. As shown below in Example 3, conjugates of a quantum dot and insulin are capable of providing active insulin to the bloodstream of a subject, as evidenced by the effect on measured blood glucose, following oral administration, without the addition of additional protective excipients. Accordingly, without being bound by theory, it is believed that conjugation of the insulin to the quantum dot disrupts the action of proteases and acids in the digestive tract, reducing degradation of the insulin before absorption, leading to the delivery of active insulin to the liver of the subject. It is envisioned that the same protective effect would be applied to other proteins and peptides conjugated with a quantum dot.

It is envisioned that a suitable pharmaceutical composition would deliver the conjugates to the intestinal tract of the subject, from where they are absorbed through the lumen of the small intestine and into the capillaries of the small intestine. Accordingly, the hydrodynamic size of the conjugates, comprising a quantum dot, insulin, and optionally a biopolymer, should be of a small enough size to allow rapid absorption of the conjugate from the intestine and into the intestinal capillaries.

Oral administration and hence an intestinal route of absorption is further advantageous and superior over other methods of absorption or administration such as injection or transdermal administration, as the blood of the small intestine capillaries proceeds directly to the liver for first-pass metabolism. Advantageously, it is preferred that the conjugates are metabolised in the liver, rather than in any other cells or organs of the subject. This is because the liver plays several important roles in maintaining glucose levels, including as a store for glucagon and a site for lipogenesis (i.e., making fatty acids from excess glucose). Further, insulin release from the liver more closely represents natural release of insulin in a healthy subject, compared to parenteral administration of an active form of insulin, which is the current preferred therapy.

Another advantage of oral administration of insulin is that the insulin when conjugated to a quantum dot and optionally coated in a biopolymer, may be further protected from the harsh conditions of the digestive tract by the biopolymer, and/or absorption through the intestinal wall may be improved, depending on the characteristics of the biopolymer.

Another advantage of oral administration is that the conjugates may be restrained from, or limited from, entering the systemic system of the subject. As shown in Example 1, the majority of uncoated quantum dots described herein are absorbed quickly after administration and accumulate in the liver, before being eliminated by biliary excretion, thereby avoiding entering the systemic circulatory system of the subject. It is also shown herein that the biopolymer coating, where applied, can affect the distribution of conjugates in the subject, further increasing absorption and accumulation by the liver.

### Treatment of Diabetes

The conjugates of the present invention may be used in a method of treating diabetes in a subject, particularly with reference to Example 4 (insulin) or Example 6 (liraglutide). The treatment may be curative and may include administration of insulin to lower blood glucose in a subject that may be hyperglycaemic. The treatment may be preventative or prophylactic in that the diabetic subject may have normal blood glucose levels at administration, but it may be expected that the subject will become hyperglycaemic soon after administration of the conjugates. For example, the conjugates may be taken orally before or with food that may be expected to increase the blood glucose levels of the subject, particularly if the food is high in carbohydrates. The diabetes to be treated may be type I diabetes, or it may be type II diabetes.

The treatment may include administration of a composition comprising conjugates of the present invention, whereby insulin as released into the blood stream of the subject after metabolism of the conjugates by the liver. By “metabolism” it is meant that cleavage of the conjugate, so as to form at least one insulin molecule and a quantum dot, occurs in the cell with the insulin being released into the blood stream and the quantum dot excreted via the biliary system. Administration may be by any suitable route, such as parenteral administration, for example subcutaneous injection, or it may be by oral administration, or it may be by transdermal administration. Preferably, the route of administration is by oral administration. The composition may include pharmaceutically acceptable excipients that are suitable for, and hence dependent on, the route of administration. For example, suitable excipients for oral administration may not be suitable of parenteral administration.

The dose of insulin or liraglutide provided by the conjugates may be the same as, or higher than, the dose of insulin or liraglutide provided by conventional parenteral therapy. For humans, the standard dose of insulin is 1 International Unit (IU), which is defined as 0.0347 mg. The dose of insulin administered to the subject may be between about 0.1 IU/kg and about 100 IU/kg, or it may be between 0.1 IU/kg and 15 IU/kg, 0.5 IU/kg and 20 IU/kg, 1 IU/kg and 10 IU/kg, 5 IU/kg and 25 IU/kg, 15 IU/kg and 30 IU/kg, 20 IU/kg and 50 IU/kg, 25 IU/kg and 75 IU/kg, 40 IU/kg and 80 IU/kg, 30 IU/kg and 70 IU/kg, 10 IU/kg and 90 IU/kg, 1 IU/kg and 99 IU/kg or 2.5 IU/kg and 12 IU/kg, e.g., about 0.1, 0.2, 0.3, 0.4, 0.5, 0.6, 0.7, 0.8, 0.9, 1, 1.5, 2, 2.5, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or 100 IU/kg. The dose of liraglutide administered to the subject may be between 100 μg/kg and about 3000 μg/kg, or it may be between about 200 μg/kg and about 2500 μg/kg, or it may be between about 1250 μg/kg and 2500 μg/kg, e.g., about 100, 150, 200, 250, 300, 350, 400, 450, 500, 600, 700, 800, 900, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950 or 3000 μg/kg. The dose of oral QD-insulin conjugate required to achieve a therapeutic effect may be the same or higher than the dose required to achieve a therapeutic effect of subcutaneous insulin (SC-insulin). The ratio of therapeutic dose of SC-insulin to oral QD-insulin may be between about 1:1 and about 1:50, or it may be between about 1:5 and about 1:40, or between about 1:10 and 1:25, or it may be 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49 or 1:50.

The desired therapeutic effect of administration of the conjugates described herein is the lowering of the blood glucose in the subject. This therapeutic effect may be evident shortly after administration, such as between about 5 minutes to about 90 minutes after administration, or between about 5 to 30 minutes, 10 to 25 minutes, 10 to 15 minutes, 15 to 45 minutes, 20 to 50 minutes, 30 to 90 minutes, 40 to 80 minutes, 10 to 75 minutes, 60 to 90 minutes, 50 to 85 minutes or 30 to 60 minutes after administration, e.g., 5, 6, 7, 8, 9, 10, 15, 20, 25, 30, 35, 40, 45, 50, 55, 60, 65, 70, 75, 80, 85 or 90 minutes after administration. This therapeutic effect may be a long-acting effect or a short-acting effect. By “short-acting”, it is meant that the therapeutic blood glucose lowering effect is observed in the subject for a period of up to about 2 hours after administration, or up to about 1.5 hours after administration, or up to about 1 hour after administration. By “long-acting”, it is meant that the therapeutic blood glucose lowering effect may be observed in the subject for between about 2 hours and about 8 hours after administration, such as between 2 hours and 4 hours, 4 hours and 8 hours, or 3 hours and 6 hours, e.g., about 2, 2.5, 3, 3.5, 4, 4.5, 5, 6, 7 or 8 hours after administration. The length of observable activity may be influenced by several factors, for example the composition and/or thickness or the biopolymer coating, the hydrodynamic diameter of the conjugate, the time of day of administration, the excipients of the composition administered, or a combination of these factors.

### Other Therapeutic Proteins and Peptides

In the above description and examples provided below, insulin has been used to demonstrate the effectiveness of attachment of a protein- or peptide-based therapeutic with quantum dots of the present invention, in order to provide a dosage form of a protein- or peptide-based therapeutic which maintains effectiveness when administered orally to a subject. However, a skilled person would readily expect that, by using the same techniques described herein, other proteins or peptides that are effective therapeutically, but not orally bioavailable, may also be suitable for conjugation with a quantum dot for oral administration. In particular, proteins and peptides that are effective when delivered directly to the liver, small bowel, pancreas, kidneys or other organs of the gastrointestinal tract of the subject, yet are not currently orally bioavailable, may also be able to be delivered to a subject in an oral dosage form using the present invention.

Proteins and peptides suitable for conjugation with the quantum dots described herein may be any therapeutically-active protein or peptide, up to about 30 kilodaltons (kDa) in size, e.g., it may be about or up to 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 kDa in size. In order to attach the protein or peptide to the QD surface via EDC/NHS, they also require a primary amine group available. Non-limiting examples of proteins and peptides of this size and comprising a primary amine suitable for conjugation with quantum dots for oral administration, as described herein, may be, for example, insulin (e.g., 51 amino acids; about 6 kDa) and analogues or derivatives thereof, growth hormone (e.g., 191 amino acids; about 22 kDa), glucagon-like peptide-1 (GLP-1) agonists such as liraglutide (30 amino acids; about 4 kDa) and exenatide (39 amino acids; about 4 kDa), glucagon-like peptide-2 (GLP-2) agonists such as apraglutide (33 amino acids; about 4 kDa), platelet derived growth factor (PDGF) beta receptor modulators such as BOT191 (Fibroferon) (about 9 kDa), or integrin alpha-4/beta-7 antagonists such as PN-10943, vasopressin, interleukins (less than 30 kDa in size), enkephalins, endorphins and the like. These therapeutically effective proteins and peptides above may be used in the treatment of type I or type II diabetes (insulin and GLP-1 agonists); obesity (liraglutide); growth hormone deficiency, particularly in subjects in old age (growth hormone); diabetic nephropathy, liver fibrosis, NASH or renal fibrosis (BOT191); celiac disease and other gastrointestinal diseases (apraglutide); or inflammatory bowel disease or ulcerative colitis (PN-10943). The above peptides and proteins are only examples of current therapeutics that are not currently orally bioavailable but would be advantageous to a subject to be able to self-administer orally.

### EXAMPLES

The invention disclosed herein may be better understood with reference to the following examples, which are not intended to be limiting.

### Example 1— Method of Forming Quantum Dot-Insulin Conjugates

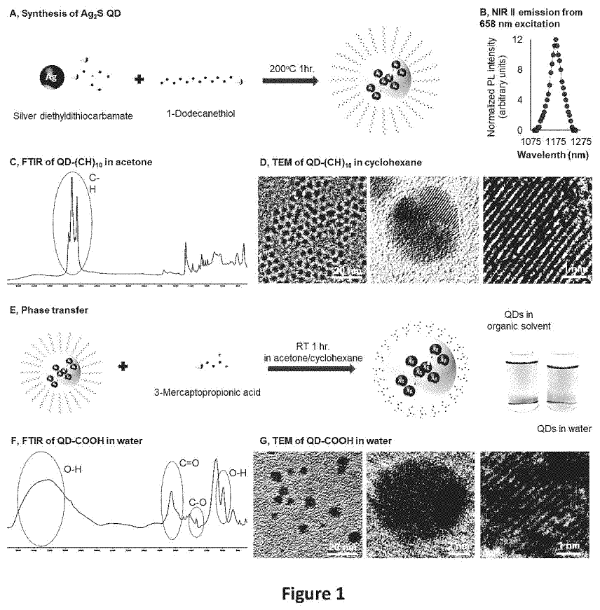

The Ag₂S quantum dots are prepared in a process as set out in FIG. 1. Essentially, silver diethyldithiocarbamate is mixed with 1-dodecanethiol and incubated at 200° C. for 1 hour to produce QDs that are soluble in cyclohexane. Characterisation of the QDs produced show a near infrared emission at 1175 nm from 658 nm excitation (FIG. 1b) and an FTIR spectra indicating C—H bonds on the surface (FIG. 1c). To produce water-soluble QDs, a phase transfer reaction is then carried out, where the QDs are incubated with 3-mercaptopropionic acid in acetone/cyclohexane at room temperature for 1 hour (FIG. 1e). Further FTIR analysis of these water-soluble QDs show bonds indicative of carboxylic acid groups on the surface, to which proteins or peptides can be conjugated (FIG. 1f).

The conjugation of QDs and insulin are performed using EDC/NHS followed by assembly of the chitosan with galactose or glucose biopolymer. QDs are incubated at room temperature with EDC and NHS for 1 hr at pH 5-6 under mixing conditions. The pH is then changed to pH 9-10 using either NaCO₃ buffer or NaOH. Insulin is then added to the solution under mixing conditions and incubated for 4-6 hrs. The solution is then dialysed (3500 or 10000 kDa cut off tubing) in water for 2 hrs, 4 hrs and overnight (16 hrs). The solution is mixed with chitosan with galactose or glucose biopolymer at room temperature for 0.5-4 hrs. The solution is then dialysed (3500 or 10000 kDa cut off tubing) in water for 2 hrs, 4 hrs and overnight (16 hrs).

Chitosan with galactose or glucose biopolymer is produced from a mixture of chitosan with galactose or glucose (ratio may be 1:1000 to 1000:1 [ratio is 1:2]). Chitosan is prepared using 1% acetic acid at 70° C. for 1-4 hrs and filtered (1 urn filter). Linking between chitosan and galactose or glucose occurs using gradual controlled cooling from 70 to 20° C. over 1-2 hrs. Purification of material is performed using ethanol solutions (50-100%) and centrifugation (chitosan with galactose or glucose biopolymer forms a gel while unconjugated materials are dissolved in ethanol).

The method above describes the conjugation of QDs and insulin, however it is envisioned that any therapeutic protein or peptide could be used in the place of insulin in this example, in order to produce therapeutic QD conjugates for treating a range of diseases.

### Example 2— Uptake of Insulin-Quantum Dot Conjugate

Conjugates of radiolabeled insulin and quantum dots were produced in a method as described above, albeit with radiolabeled insulin used instead of native insulin. Two groups of healthy rodents were then orally administered either a dose of conjugates or equivalent radiolabeled unbound insulin. The distribution of radioactivity was then monitored 30 minutes after administration.

Approximately 50% of the radioactive label was found in the liver of the rodents 30 minutes after oral administration, compared with approximately 1% of the unbound radiolabeled insulin after 30 minutes. This indicates that the conjugates of the present invention are capable of providing insulin to the liver via an oral route for more effectively than without conjugation and that conjugation has a protective effect in maintaining insulin effectiveness in the digestive tract.

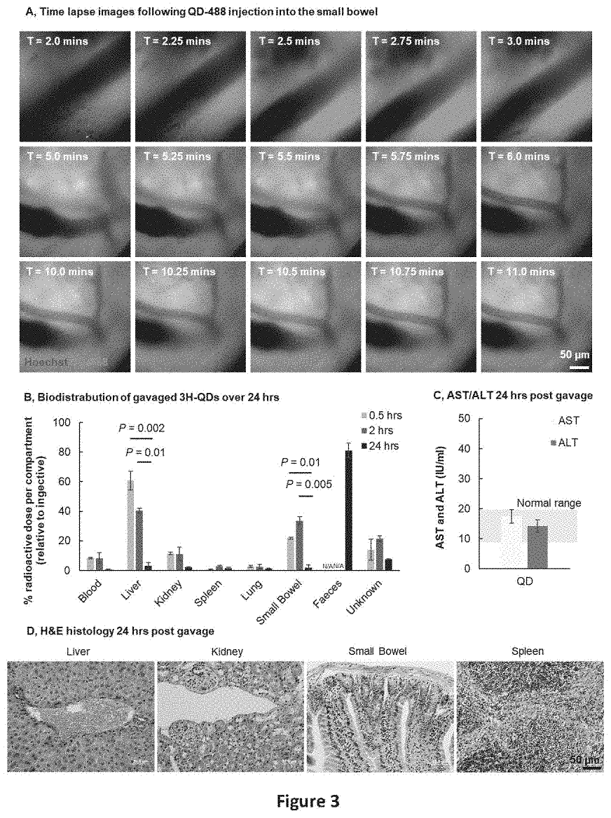

However, the preference for accumulation in the liver of the QD-insulin conjugates is not a function of the insulin, but rather a feature of the QD coated with a biopolymer. This is demonstrated in FIG. 3, whereby ³H-labelled QDs without a protein conjugated to it are administered orally into a mouse model, resulting in about 60% accumulation in the liver 30 minutes after administration. In other words, any protein or peptide that can be conjugated to the Ag₂S QDs can be delivered to the liver following oral administration.

### Example 3— Glucose Tolerance Test with an Oral Dose of Conjugate

A population of 30 healthy rodents without diabetic symptoms were obtained, and were divided into six treatment groups: one group of 5 rodents received 2 IU/kg of insulin by intraperitoneal injection 30 minutes prior to a glucose gavage; one group received 100 IU/kg of insulin by via oral gavage 30 minutes prior to a glucose gavage; one group of 5 rodents were given a dose of quantum dot-insulin conjugates equivalent to 100 IU/kg insulin 30 minutes prior to a glucose gavage; one group of 5 rodents were given a dose of quantum dot-insulin conjugates equivalent to 10 IU/kg insulin 30 minutes prior to a glucose gavage and one group of five rodents were given only a glucose gavage. Blood glucose of all rodents were then monitored over 90 minutes post-glucose gavage.

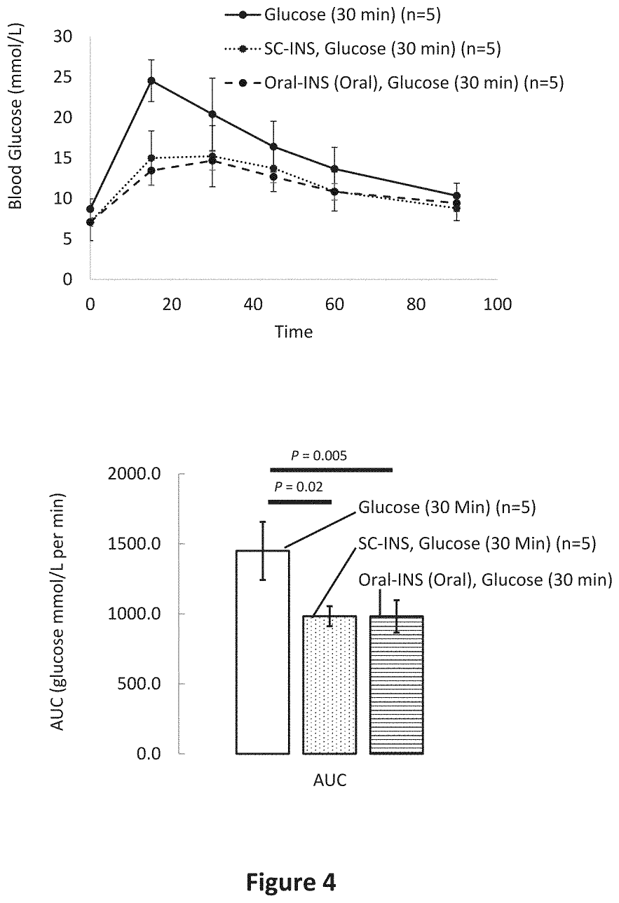

As seen in FIG. 4, the rodents in the control group that did not receive a therapy saw a rapid increase in blood glucose levels 15 minutes after receiving the glucose, with a gradually reduction over the length of the study. Contrarily, neither the 2 IU/kg insulin given by intraperitoneal injection group or quantum dot insulin group recorded a spike in blood glucose levels, indicating that both intraperitoneal injection of insulin (the current preferred therapy) and the quantum dot-insulin conjugates of the present invention, were both effective in lowering blood glucose.

Most surprising is that the orally administered conjugates displayed equal, if not better, blood glucose lowering effects than the injected insulin. This indicates that the quantum dot conjugates of the present invention are capable of delivering insulin to the bloodstream of a subject via oral administration and that the insulin is absorbed in an effective state that is capable of affecting the blood glucose levels of the subject receiving the oral dosage form of insulin.

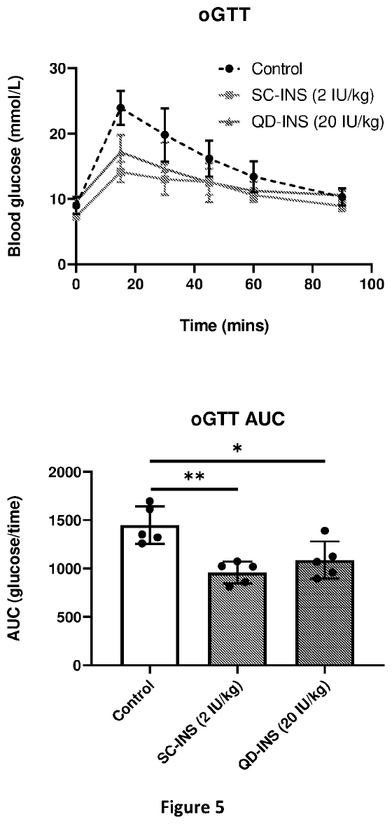

A further study was carried out on healthy rodents without diabetic symptoms comparing a saline subcutaneous injection (control), a subcutaneous injection of insulin (SC-INS, 2 IU/kg), or an oral gavage of oral-insulin (QD-INS, 20 IU/kg) 30 minutes before a glucose tolerance test (oGTT). As shown in FIG. 5, the oral QD-insulin therapy was as effective on lowering blood glucose levels in the mice as the injected insulin.

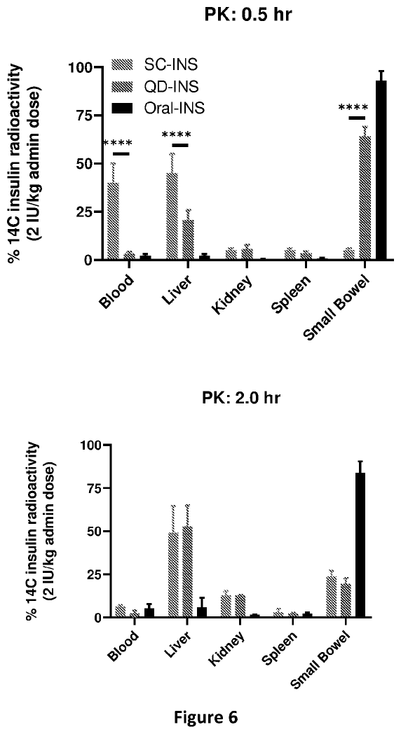

Pharmacodynamics of the QD-insulin conjugates were also investigated using radiolabelled insulin. Three mice treatment groups were fasted for 4 hours before given either an oral gavage of insulin (Oral INS), a subcutaneous injection of insulin (SC-INS) or an oral gavage of QD-insulin conjugate (QD-INS) at 2 IU/kg doses. As shown in FIG. 6, after 30 minutes the orally administered therapies were mainly located in the small bowel whilst the subcutaneously injected insulin was found in the liver and blood. However, after 2 hours, whilst the oral insulin was still located in the bowel, about 50% of the radiolabelled insulin for both the SC-INS and the QD-INS was located in the liver. In fact, the distribution of insulin between SC-INS and QD-INS was almost identical. This shows that oral QD-peptide conjugate administration results in a biodistribution after administration that mimics invasive subcutaneous administration.

### Example 4— Insulin Tolerance Test in Diabetic Mouse Model

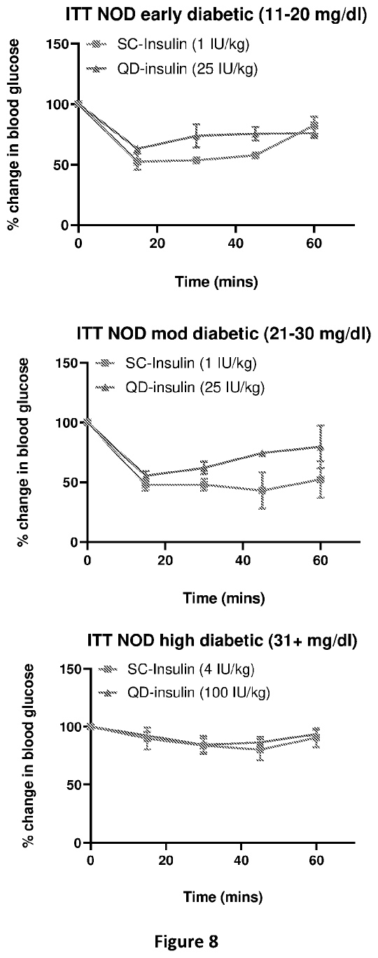

The above examples demonstrate the glucose-lowering effect of oral QD-insulin conjugates on healthy mice models. Similar experiments were also conducted on a NOD/Scid mouse model which demonstrated the development of type I diabetes with increasing blood glucose concentrations. Blood glucose was measured used a handheld Glucometer using Accu-check proforma strips. Blood was collected by tail vein sampling following a tail snip. Blood glucose was collected at −15, 0, 15, 30, 45 and 60 mins after a subcutaneous (SC) insulin injection (0.1, 1 or 2 IU/kg) or a gavage of QD-insulin conjugates (10, 20, 30, 40, 50, 75 or 100 IU/kg). As shown in FIG. 8, a QD-insulin dose about 25 times higher than the SC-insulin dose resulted in similar glucose reductions in a diabetic model.

### Example 5— Method of Forming Quantum Dot-Liraglutide Conjugates

Liraglutide was conjugated to QDs via EDC/NHS coupling similarly to insulin described above (see Example 1). 1 mM Ag₂S QD was mixed with 1 mM EDC and 1 mM NHS in a reaction vial under heavy mixing for 1 hr. Following this, the pH was altered to 9.0 using 0.5M NaCO₃ and 1-10 mM Liraglutide was added to the solution. The solution was mixed overnight and transferred to dialysis tubing 10,000 MWCO and dialyzed with 1 ml solution per 1L of MQ for 2, 4, 16 hr 4° C. in the dark. Polymer attachment to the QD-liraglutide was also carried out using the same process above in Example 1.

### Example 6— Oral Glucose Tolerance Test for Liraglutide

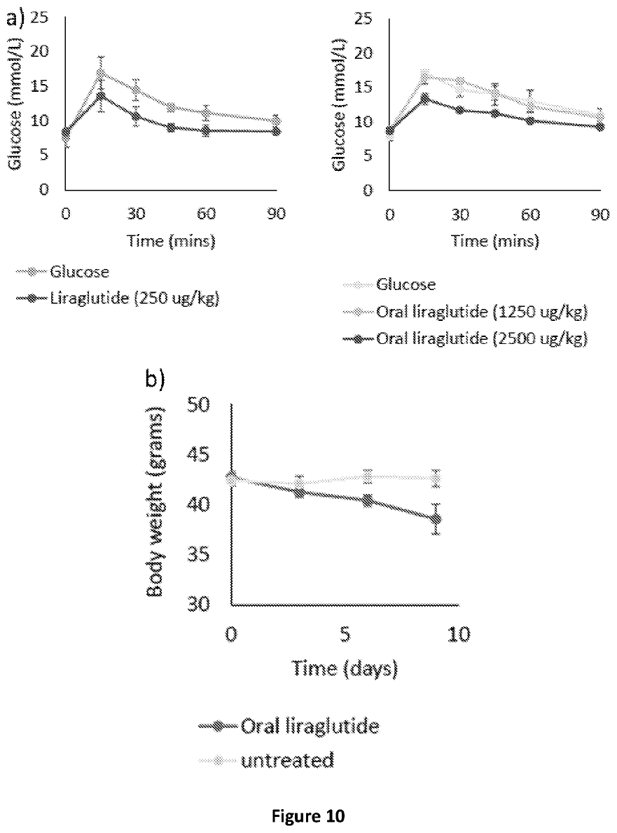

oGTTs were performed following a 4 hr fast in C57/B16 mice (see FIG. 10). Blood glucose was measured used a handheld Glucometer using Accu-check proforma strips. Blood was collected by tail vein sampling following a tail snip. Blood glucose was collected −30, −15, 0, 15, 30, 45, 60 and 90 mins after an oral bolus of 2 g/kg glucose solution. At time −120 min mice were given a subcutaneous (SC) Liraglutide injection (250 ug/kg in saline), a gavage of QD-Liraglutide-CS/GS (2500 ug/kg in water) or were untreated. Pharmacodynamics were determined based on the effects of liraglutide treatment on the area under the curve (AUC) of individual oGTTs experiments. For pharmacodynamic measurements effect of SC-liraglutide and QD-liraglutide was determined relative to the reduction in the AUC.

### Example 7—Other Therapeutics

The Examples provided above are directed to conjugates of insulin and liraglutide, partially due the ease of measurement of the therapeutic activity of these therapeutics on blood glucose concentrations. In other words, insulin provides a good ‘proof of concept’ model to determine that the proteins and peptides administered orally in such conjugates are protected and are still viable or capable of imparting a therapeutic effect after absorption and hepatic transport. However, it is expected that other proteins or peptides would also be viable if conjugated to Ag₂S QDs and coated in a biopolymer.

To determine if other proteins or peptides are therapeutically effective after conjugation and oral delivery to the liver, the skilled person would be able to (1) devise assays, (2) measure physiological effects of the target therapeutic peptide or protein or blood components with mass spectrometry, or (3) radiolabel the peptide or protein to determine if the peptide or protein is being delivered to the organ of most therapeutic interest (such as the liver, pancreas or small intestine) and how long after administration before this occurs. For instance, if the peptide or protein is intended to treat a condition associated with inflammation (for example, celiac disease or inflammatory bowel disease), it may be possible to measure blood markers associated with inflammation (e.g., IL-113, IL-6, IL-8 (MIP-2), IFNγ, TFNα, etc.).

One example includes comparing oral administration of a conjugate comprising the Ag₂S QDs and apraglutide (or other GLP-2 agonist) to a subcutaneous injection of the same therapeutic peptide in a mouse model of celiac disease, similar to the comparative studies described above. After administration of the GLP-2 agonist, the treated mice may be given an oral gavage of gluten and various blood markers, such as tTG-IgA, compared.

Another example includes comparing a subcutaneous injection of human growth hormone to a therapeutic dose administered as a conjugate of QDs and human growth hormone, similar to the comparative studies described above. The human growth hormone may be measured directly by a well-known assay to determine blood levels of the hormone. The effectiveness of the administered hormone may also be determined by measuring IGF-1 (insulin growth factor-1) in the blood, and/or by observing and comparing the growth of treated versus non-treated subjects (such as mice).

Assays for measuring the effects of other therapeutic peptides and proteins would be able to be developed by the skilled person, based on their knowledge of the particular disease processes and the therapeutic effects of the protein or peptide that is expected.

1. A composition comprising a therapeutic amount of a conjugate, wherein the conjugate comprises a quantum dot and a therapeutically effective peptide or protein.

2. The composition of claim 1, wherein the peptide or protein is less than about 30 kDa in size.

3. The composition of claim 1 or claim 2, wherein the peptide or protein is selected from the group consisting of insulin, growth hormone, fibroblast growth factor 21 (FGF21) a glucagon-like peptide-1 (GLP-1) agonist, a glucagon-like peptide-2 (GLP-2) agonist, a platelet derived growth factor (PDGF) beta receptor modulator, and an integrin alpha-4/beta-7 antagonist, a PYY(3-36) analogue, vasopressin, interleukins (less than 30 kDa in size), enkephalins, endorphins and combinations thereof.

4. The composition of claim 3, wherein:
the GLP-1 agonist is liraglutide or exenatide; and/or
the GLP-2 agonist is apraglutide; and/or
the PDGF beta receptor modulator is BOT191; and/or
the integrin alpha-4/beta-7 antagonist is PN-10943.

5. The composition of any one of claims 1 to 4, wherein the composition is formulated for oral administration.

6. The composition of any one of claims 1 to 5, wherein the quantum dot is an Ag₂S quantum dot.

7. The composition of any one of claims 1 to 6, wherein the average diameter of the quantum dot is between about 1 nm and about 20 nm.

8. The composition of claim 7, wherein the average diameter is less than 10 nm.

9. The composition of any one of claims 1 to 8, wherein the conjugate further comprises a polymer.

10. The composition of claim 9, wherein the polymer is a biopolymer.

11. The composition of claim 10, wherein the biopolymer is selected from the group consisting of heparin, gelatin, hyaluronic acid, chitosan, galactose, glucose, and any combination thereof.

12. The composition of claim 11, wherein the biopolymer is chitosan, galactose, glucose, or any combination thereof.

13. The composition of any one of claims 9 to 12, wherein the polymer or the biopolymer covers at least part of the conjugate.

14. The composition of any one of claims 1 to 13, wherein the conjugate accumulates on/in a hepatocyte of the subject after oral administration.

15. The composition of any one of claims 1 to 14, wherein the composition is for administration to a subject that has been diagnosed with a condition related to insufficient endogenous peptide or protein production.

16. The composition of claim 15, wherein the condition is type I or type II diabetes.

17. The composition of any one of claims 1 to 14, wherein the composition is for administration to a subject that has been diagnosed with a condition requiring exogenous therapeutic peptide or protein administration.

18. The composition of claim 17, wherein the condition is selected from diabetic nephrology, liver fibrosis, non-alcoholic steatohepatitis (NASH), renal fibrosis, celiac disease, inflammatory bowel disease (IBD), ulcerative colitis or another gastrointestinal disease.

19. The composition of any one of claims 1 to 18, wherein the composition comprises a pharmaceutically acceptable excipient.

20. A method of treating hyperglycemia in a subject in need thereof, the method comprising administering to the subject a therapeutic amount of a conjugate comprising a quantum dot and insulin.

21. A method of treating insufficient endogenous peptide production in a subject in need thereof, the method comprising administering to the subject a therapeutic amount of a conjugate comprising a quantum dot and a protein or peptide effective in replacing the insufficient endogenous peptide.

22. A method of treating a subject suffering from a condition, wherein the condition is treatable with administration of a therapeutic exogenous peptide or protein, the method comprising administering to the subject a therapeutic amount of a conjugate comprising a quantum dot and the therapeutic exogenous peptide or protein.

23. The method of any one of claims 20 to 22, wherein the conjugate is administered to the subject orally.

24. The method of claim 21, wherein the condition is type I or type II diabetes.

25. The method of any one of claims 20 to 24, wherein the quantum dot is a Ag₂S quantum dot.

26. The method of any one of claims 20 to 25, wherein the average diameter of the quantum dot is between about 5 nm and about 20 nm.

27. The method of any one of claims 20 to 26, wherein the conjugate further comprises a polymer.

28. The method of claim 27, wherein the polymer is a biopolymer.

29. The method of claim 28, wherein the biopolymer at least partially coats the conjugate.

30. The method of claim 28 or claim 29, wherein the biopolymer is selected from the group consisting of heparin, gelatin, hyaluronic acid, chitosan, galactose, glucose, and any combination thereof.

31. The method of claim 30, wherein the biopolymer is chitosan, galactose, glucose, or any combination thereof.

32. A method of delivering a peptide or protein to an organ of a subject, the method comprising orally administering a conjugate comprising a quantum dot and the peptide or protein to the subject, wherein the organ is selected from the liver, pancreas, small bowel or kidneys.

33. The method of claim 32, wherein the quantum dot is an Ag₂S quantum dot.

34. The method of claim 33, wherein the Ag₂S quantum dot is between about 5 nm and about 20 nm in diameter.

35. The method of any one of claims 32 to 34, wherein the conjugate optionally comprises a biopolymer selected from the group consisting of gelatin, chitosan, galactose, glucose, and any combination thereof.

36. A method of lowering blood glucose in a subject, the method comprising orally administering a conjugate comprising a quantum dot and insulin to the subject, wherein the quantum dot is an Ag₂S quantum dot between about 5 nm and about 20 nm in diameter, wherein the conjugate is metabolized in a hepatocyte, thereby releasing the insulin into the blood stream of the subject.

37. Use of a conjugate comprising a quantum dot and insulin for the manufacture of a medicament effective in the treatment of type I or type II diabetes.

38. Use of a conjugate comprising a quantum dot and a protein or peptide in the preparation of a medicament.

39. The use of claim 38, wherein the medicament is for treating insufficient endogenous production of the peptide or protein in a subject.

40. The use of any one of claims 37 to 39, wherein the medicament is formulated for oral administration.

41. The use of any one of claims 37 to 40, wherein the conjugate further comprises a polymer or a biopolymer.
